(19) United States
(12) Patent Application Publication
(10) Pub. No.: US 2012/0193255 A1
(43) Pub. Date: Aug. 2, 2012

(54) MEDICAL DEVICE AND PACKAGING INTERFACE

(76) Inventors: Raymond J. Lareau, Westford, MA (US); Ryan Heller, New Bedford, MA (US); Jon T. McIntyre, Newton, MA (US); Paul DiCarlo, Middleboro, MA (US); Mark Girard, Medway, MA (US)

(21) Appl. No.: 13/017,916

(22) Filed: Jan. 31, 2011

(51) Int. Cl. A61B 19/02

(52) U.S. Cl. 206/364

(57) ABSTRACT

A medical device packaging comprising a connector coupled to a longitudinal element having a lumen sized and shaped to receive an shaft of a medical device extending distally from a hub of the elongate member. The connector including a first branch having a lumen open to the lumen in the elongate member and a second branch having lumen open to the lumen in the first branch. A proximal end of the first branch being configured to mate with a distal end of a housing of the medical device to be received in the packaging. The hub of the medical device including laterally extending wings having opposing contours. The medical device including a strain relief member coupled at a distal end of the hub of the medical device.

### FIELD

The embodiments presented herein relate generally to medical devices with elongate shafts, and more particularly, to packaging that facilitates flushing of the elongate shaft.

### BACKGROUND

A catheter is a medical device that may be inserted into a body structure to deliver fluids from an external source to an internal body structure. A catheter generally comprises a flexible shaft extending longitudinally from a proximal end to a distal end and often includes a hub at the proximal end for the connection of other devices or fluid conduits. Catheters, such as microcatheters, designed to thread deep into smaller body structures (e.g., the vascular system, the biliary tree, etc.) are often especially long to enable them to pass through other devices and a succession of vessels to reach target smaller vessels. Such smaller catheters are often more delicate and must be carefully packaged to prevent damage thereto.

Catheter packaging may includes a long tubular structure designed to house a length of the catheter shaft therein. The tubular structure may take a variety of different shapes such as for example, straight, curved or hooped. Portions of the tubular structure may be fixed to one another such that the tubular element is retained in a particular shape, which may be determined, for example, by the length of the catheter. Some catheters may require hydration and/or activation of a coating prior to use which necessitates flushing the catheter with fluid. For example, the catheter may be treated with a hydrophillic coating which provides lubrication to facilitate insertion when activated by water, saline or body fluid. However, current catheter packaging often does not facilitate effective flushing of fluid along an entire packaged length of the catheter.

### SUMMARY

The embodiments described herein are directed to packaging for a medical device such as a catheter that facilitates flushing of the packaged length of the catheter. The packaging comprises a longitudinal element defining a first lumen sized and shaped to receive therein a shaft of an elongate medical device such as a catheter with a desired clearance around an outer diameter of the shaft of the medical device. The length of the longitudinal element can be selected to receive a preferred packaged length of the shaft of the elongate medical device extending distally from a hub at a proximal end thereof. The packaging further comprises a connector attached to the proximal end of the longitudinal element. The connector, which may be a Y- or T-shaped connector, a manifold, or the like, includes a first branch defining therein a second lumen open to a proximal end of the first lumen and extending substantially along a longitudinal axis of the longitudinal element and a second branch defining therein a third lumen open to the second lumen and extending away from the longitudinal axis at a predetermined angle. A proximal end of the first branch including a structure configured to mate with a corresponding feature on a distal end of a housing or hub of the medical device to be received in the packaging to form a substantially fluid tight connection therebetween such that fluid introduced into the third lumen is prevented from exiting the proximal end of the second lumen and is forced move distally through the first lumen to the distal end of the longitudinal element without exiting from the distal end.

In one embodiment, the hub may act as a handle for maneuvering the elongate shaft of the medical device. The hub includes laterally extending wings with opposing contours. The contours of the wings allow operator fingers to interface with the wings to facilitate handling and rotation in the clockwise or counterclockwise directions.

In another embodiment, the medical device includes a strain relief member coupled to the elongate shaft and the hub. The strain relief member comprises a helical coil or spring that is covered by a polymeric tube. The tube is formed over the coil in a way that fills in the spaces between the coils resulting in the formation of multiple hinge points.

Other systems, methods, features and advantages will be or will become apparent to one with skill in the art upon examination of the following figures and detailed description. It is intended that all such additional systems, methods, features and advantages be included within this description, be within the scope of the devices, systems and methods described herein, and be protected by the accompanying claims.

### BRIEF DESCRIPTION OF THE DRAWINGS

The figures provided herein are not necessarily drawn to scale, with some components and features being exaggerated for clarity. Each of the figures diagrammatically illustrates aspects of the embodiments.

FIG. 1 shows a side view of a system according to an exemplary embodiment.

FIG. 2 shows a side view of a Y-connector according to the system of FIG. 1.

FIG. 3 shows a top view of a hub according to the system of FIG. 1.

FIGS. 4a and 4b show opposing side views of the hub of FIG. 3.

FIG. 5 shows a side view of a retaining clip coupled to the Y-connector and the hub of the system of FIG. 1.

FIG. 6 shows a side view of an exemplary embodiment of a medical device.

FIGS. 7a show partial detail views of the medical device in FIG. 6 taken along line 7.

It should be noted that elements of similar structures or functions are generally represented by like reference numerals for illustrative purpose throughout the figures. It should also be noted that the figures are only intended to facilitate the description of the preferred embodiments.

### DETAILED DESCRIPTION

The embodiments provided herein may be further understood with reference to the following description and the appended drawings, wherein like elements are referred to with the same reference numerals. The embodiments provided herein relate to medical device packaging. In particular, exemplary embodiments described herein include a catheter and a packaging interface which allows fluid to be flushed along an enclosed length of the catheter within the packaging. Specifically, exemplary embodiments described herein include a connector attached to a packaging that is coupleable with a hub of a catheter and creates a fluid path through the packaging that is biased by flow dynamics.

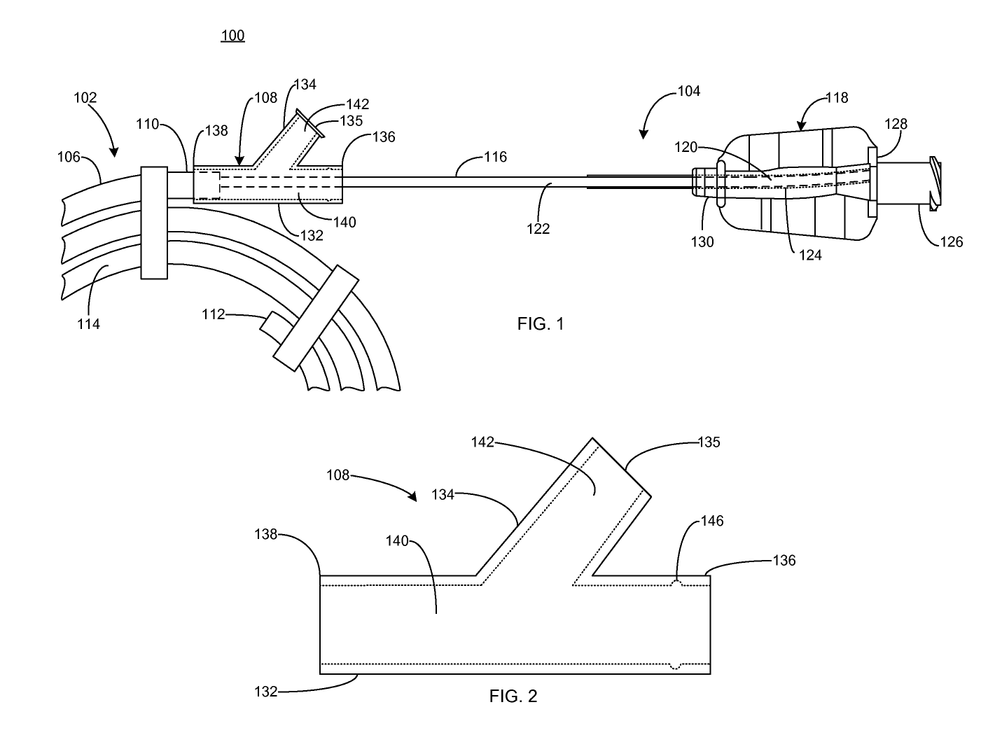

As shown in FIG. 1, a system 100 according to a first exemplary embodiment comprises a packaging 102 and a catheter 104. The packaging 102 includes an elongate longitudinal element 106 and a connector 108, which, as depicted, is configured as a Y-connector. The connector 108 can also be a T-connector, a manifold, and the like. The longitudinal element 106 extends from a proximal end 110 to a distal end 112 and includes a lumen 114 extending there through for accommodating a length of the catheter 104. The Y-connector 108 is attached to the proximal end 110 of the longitudinal element 106. The catheter 104 includes a longitudinally extending flexible shaft 116 and a hub 118 attached adjacent to a proximal end 120 of the flexible shaft 116. The flexible shaft 116 can have various cross-sectional geometries including round, oval and the like. It will be understood by those of skill in the art that the catheter 104 may be any standard catheter used to deliver fluid to and/or remove fluid from an internal body structure via a lumen 122 of the flexible shaft 116. The Y-connector 108 and the hub 118 are detachably coupleable, creating a fluid-tight, or at least near or substantially fluid tight connection that may be decoupled once the longitudinal element 106 and the flexible shaft 116 have been properly flushed. The connection between the Y-connector 108 and the hub 118 can be made near or substantially fluid tight in instances in which the lubricity of the flush fluid is used to aid in decoupling the hub 118 from the Y-connector 108.

The longitudinal element 106 of the packaging 102 is also preferably flexible permitting the longitudinal element 106 to be maintained in any of a variety of shapes and/or configurations. For example, in the embodiment shown, the longitudinal element 106 is maintained in a hoop configuration with a length of the longitudinal element 106 coiled upon itself permitting a greater length of the longitudinal element 106 to be accommodated in a reduced space facilitating storage and/or transport. It will also be understood by those of skill in the art that the longitudinal element 106 may be maintained in any configuration, so long as the longitudinal element 106 accommodates a predetermined packaged length of the flexible shaft 116. In instances where the flexible shaft 116 does not include a curved distal end, the packaged length may include the entire length of the flexible shaft 116. In instances where the flexible shaft 116 includes a curved distal end, the packaged length may include a predetermined length that is less than the entire length of the flexible shaft 116.

The Y-connector 108, which is attached to the proximal end 110 of the longitudinal element 106, includes, as shown in FIG. 2, a first portion 132 forming a first stem thereof and a second portion 134 forming a second stem to form the Y-shape. The first portion 132 extends from a proximal end 136 to a distal end 138 and includes a lumen 140 extending there through. The lumen 140 may be substantially aligned with the lumen 114 of the longitudinal element 106, facilitating insertion of the flexible shaft 116 of the catheter 104 into and there through. The second portion 134 extends from the first portion 132 at an acute angle of about 15° to 90° relative to a longitudinal axis of the first portion 132, between the proximal and distal ends 134, 138. The second portion 134 includes a port 135 separated from the first portion 132 and located proximally of the point of intersection between the first and second portions 132, 134, respectively. The second portion 134 includes a lumen 142 along a length thereof extending from a proximal end at the port 135 to a distal end open to the lumen 140 of the first portion 132. Thus, it will be understood by those of skill in the art that the second portion 134 may act as a port for injecting fluid into the Y-connector 108 and through the longitudinal element 106.

It will be understood by those of skill in the art that the flexible shaft 116 may be of a variety of lengths and diameters so long as the flexible shaft 116 is insertable into the body. The length and the diameter or cross-sectional dimensions of the flexible shaft 116 may be selected depending on a purpose of the catheter 104 and a target location to which the flexible shaft 116 will be inserted. For example, where catheter 104 is a microcatheter, the flexible shaft 116 may be very long and of a small diameter in order to intravascularly access the target location. It will also be understood by those of skill in the art that the lumen 122 of the flexible shaft 116 may be sized to track over a guidewire for insertion into the body of a patient.

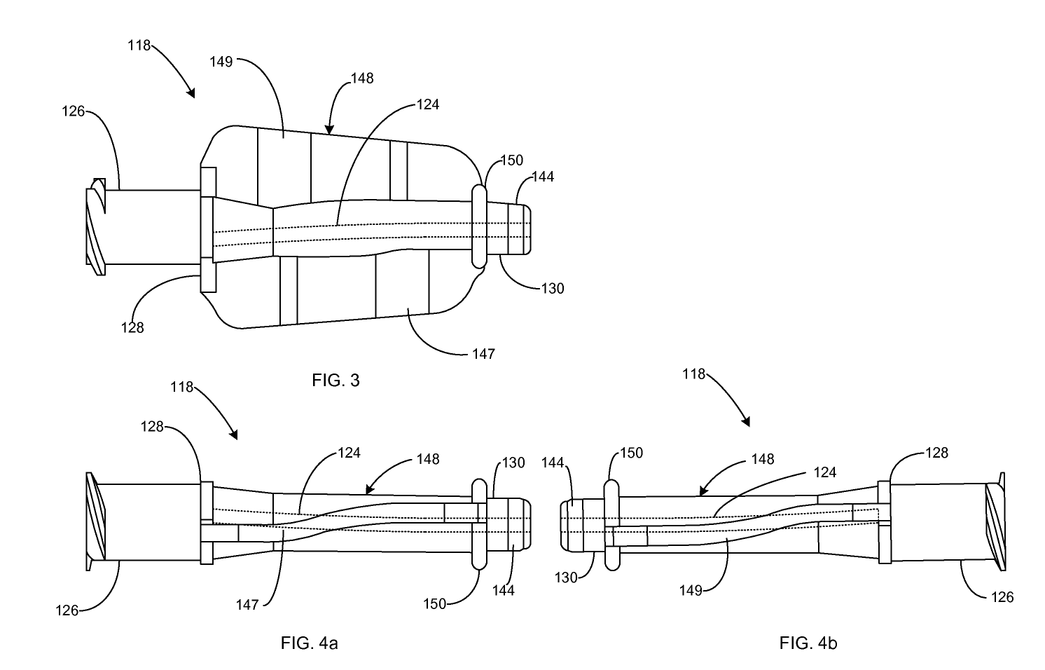

As shown in FIGS. 3-4, the hub 118 may act as a handle for maneuvering the flexible shaft 116 of the catheter 104 and/or as a proximal port for facilitating communication between the lumen 122 of the flexible shaft 116 and other medical devices used to withdraw and/or inject fluid into and/or out of the body via the catheter 104. Thus, it will be understood by those of skill in the art that the hub 118 may include a body 148 to facilitate maneuvering the flexible shaft 116 and a channel 124 for accommodating the proximal end 120 of the flexible shaft 116 such that any medical device attached to a connector 126 extending from a proximal end 128 of the body 148 of the hub 108 directs fluid through the lumen 122 of the shaft 116 between the attached medical device and a target location in the body.

In an exemplary embodiment, a configuration of the body 148 of the catheter hub 118 facilitates handling, tracking and manipulation of the device during clinical procedure. The body 148 preferably includes contoured wings 147 and 149 extending outwardly from the channel 124. As depicted in FIGS. 4a and 4b, the wings 147 and 149 are constructed with an opposing contour from the other. The contours of the wings 147 and 149 allow the operators fingers to fit into the wings 147 and 149 to facilitate handling and rotation in the clockwise, or counterclockwise directions.

The hub 118 further includes a mating element 130 at a distal end 150 of the body 148, coupleable with the Y-connector 108 to form an at least partially fluid tight connection. The distal mating element 130 may include, for example, a protrusion insertable into the proximal end 136 of the Y-connector 108. Thus, it will be understood by those of skill in the art that the diameter of the distal mating element 130 may be substantially similar to or only slightly smaller than a diameter of the lumen 140 at the distal end 136 of the Y-connector 108 so that when the hub 118 and the Y-connector 108 are coupled, the coupling produces a tight fit. It will also be understood by those of skill in the art that the distal mating element 130 may further include a feature facilitating removable coupling of the distal mating element 130 with the proximal end 136 of the Y-connector 108. For example, the distal mating element 130 may further include a protrusion 144 extending around at least a portion of a perimeter of the distal element 130 which snaps into a correspondingly shaped recess or groove 146 in the lumen 140 of the Y-connector 108. The distal element 130 may be removed from the Y-connector 108 by exerting a pulling force on either the hub 118 and/or the Y-connector 108. It will be understood by those of skill in the art, however, that the hub 118 and the Y-connector 108 may include any mating mechanism so long as the hub 118 and the Y-connector 108 are detachably coupleable in an at least partially or near fluid tight connection.

This connection forms an obstruction or “choke” preventing fluid from leaking out of the Y-connector 108 proximally toward the proximal end 120 of the shaft 116, urging fluid inserted into the port 135 through the Y-connector 108 distally along a length of the longitudinal element 106. Thus, once the hub 118 and the Y-connector 108 have been coupled, a user may inject fluid (e.g., water or saline) into the Y-connector via the port 135 of the second portion 134 passing the fluid through the lumen 142 into the lumen 140 and distally into the lumen 114 along the length of the longitudinal element 106 flushing the full length of the enclosed flexible shaft 116.

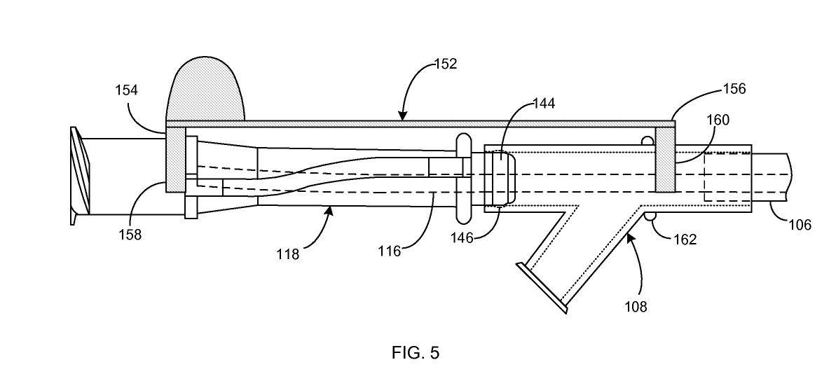

According to a further embodiment, as shown in FIG. 5, the system 100 further comprises a retention clip 152 ensuring that the hub 118 and the Y-connector 108 do not come apart during the flushing process. The retention clip 152 extends substantially longitudinally from a proximal end 154 including a first element 158 for attaching to the hub 118 and a distal end 156 including a second element 160 for attaching to the Y-connector 108. The first element 158 is sized and shaped to accommodate the connector 126 extending from the proximal end 128 of the hub 118 such that, in an operative position, the first element 158 abuts the proximal end 128 of the body 148. The second element 160 is sized and shaped to accommodate a portion 164 of the Y-connector 108 along the first portion 132, distally of the second portion 134.

An o-ring 162 or other compressible protrusion may be positioned distally about the periphery of the second portion 134 along the first portion 132 such that the second element 160 abuts the O-ring 162. Additionally, the o-ring 162 ensures compression of the hub 118 and the Y-connector 108 while allowing for tolerance ranges between the hub 118, the Y-connector 108 and the retention clip 152. A length of the retention clip 152 is preferably selected to be substantially similar to a distance from the proximal end 128 of the body 148 of the hub 118 to a point distal of the second portion 134 of the Y-connector 108 when the hub 118 and the Y-connector are in the coupled configuration. Thus, it will be understood by those of skill in the art that the o-ring 162 may be positioned according to the length of the retention clip 152 to achieve a desired compression between the hub 118 and the Y-connector 108. The compression between the hub 118 and the Y-connector 108, formed by the retention clip 152 prevents the coupling between the hub 118 and the Y-connector 108 from becoming unintentionally detached during the flushing process.

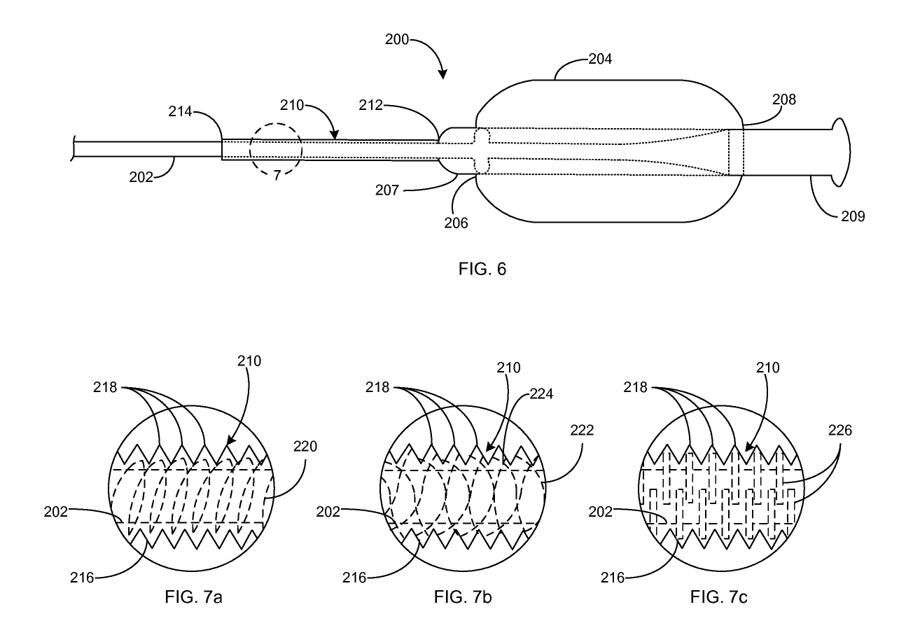

Turning to FIGS. 6 and 7, a further embodiment of a medical device includes a strain relief member 210 for a catheter device 200. As with the catheter described above, the catheter device 200 includes an elongate shaft 202 extending from a hub 204. The hub 204 includes a connector 209 extending from its proximal end 208 and a distal element 207 extending from its distal end 206. As depicted in FIG. 7a, the strain relief member 210 includes a helical coil or spring 220 that is covered by a jacket or cover 216. The jacket 216 is preferably formed from a polymeric material over the coil 220 in a way that fills in the spaces between the coils resulting in the formation of multiple hinge points 218. This can be accomplished by shrinking the polymeric tube 216 over the coil 220 or alternatively by injection molding the jacket 216 over the coil 220.

Once the jacket 216 is formed over the coil 220, the strain relief member or sub-assembly 210 is placed over the proximal end of the catheter shaft 202. A proximal end 212 of the strain relief sub-assembly 210 is coupled to the catheter shaft 202 and the distal element 207 of the hub 204 and the distal end 214 of the strain relief sub-assembly 210 is coupled to the catheter shaft 202. Coupling of the proximal end 212 of the strain relief sub-assembly 210 to the catheter shaft 202 and distal element 207 of the hub 204 is preferably done either through insert molding or adhesive bonding. Transfer of the rotational force from the hub 204 to the shaft 202 is enabled by attaching both ends 212 and 214 of the strain relief sub-assembly 210, and the transfer of rotation is further enhanced by the presence of the coil 220 in the jacket 216.

The primary function of relieving stress at the point of attachment of the shaft 202 to the hub 204 is maintained by the configuration of the strain relief sub-assembly 210. Stress relief is enhanced by the formation of the “hinge points” 212 between the coils of the coil 220.

In alternative embodiments shown in FIGS. 7b and 7c, the single coil 220 is replaced by two counter wound springs 222 and 224 (FIG. 7b) or a braid 226 (FIG. 7c).

Alternatively, the entire length of the strain relief sub-assembly 210 could be attached to the outside of the catheter shaft 202 by thermally melting the materials together or via adhesive bonding.

It will be apparent to those skilled in the art that various modifications and variations can be made in the structure and the methodology of the present invention, without departing from the spirit or scope of the invention. Thus, it is intended that the present invention cover the modifications and variations of this invention provided they come within the scope of the appended claims and their equivalents.

1. A medical device packaging comprising:
a longitudinal element defining a first lumen sized and shaped to receive therein an elongate shaft of a medical device; and
a connector attached to the proximal end of the longitudinal element, the connector including a first branch defining therein a second lumen open to a proximal end of the first lumen and extending substantially along a longitudinal axis of the longitudinal element and a second branch defining therein, a third lumen open to the second lumen and extending away from the longitudinal axis at a preselected angle, a proximal end of the first branch configured to mate with a distal end of a housing of a medical device to be received in the packaging to form a substantially fluid tight connection therebetween such that fluid introduced into the third lumen is prevented from exiting the proximal end of the second lumen and is forced distally through the first lumen to the distal end of the longitudinal element.

2. The packaging of claim 1 wherein the connector is a Y-connector.

3. The packaging of claim 1 wherein the connector is a T-connector.

4. The packaging of claim 1, further comprising a retention clip including a distal end coupled to the connector distal of the distal end of the second branch, a proximal end of the retention clip being coupleable to a housing of a medical device to be received in the packaging to prevent the housing of the medical device from being inadvertently separated from the first branch of the connector.

5. The packaging of claim 1, wherein the structure configured to mate includes a recess extending about at least a portion of a perimeter of a first port at a proximal end of the first branch of the Y-connector to form a snap fit connection with a corresponding protrusion on a distal end of a housing of a medical device to be received in the packaging.

6. The packaging of claim 4, further comprising a compressible structure extending around at least a portion of a periphery of the connector between the distal end of the retention clip and the distal end of the second branch.

7. The packaging of claim 1, wherein the second branch extends away from the first branch at an angle selected so that a proximal end of the second branch is closer to the proximal end of the first branch than is the distal end of the second branch.

8. A packaging system comprising:
a catheter including an elongated shalt extending from a hub at a proximal end thereof to a distal end, the hub including a first mating structure at a distal end thereof;
a longitudinal element defining a first lumen sized and shaped to receive the shaft of the catheter; and
a connector attached to the proximal end of the longitudinal element, the connector including a first branch defining therein a second lumen open to a proximal end of the first lumen and extending substantially along a longitudinal axis of the longitudinal element and a second branch defining therein a third lumen open to the second lumen and extending away from the longitudinal axis at a preselected angle, a proximal end of the first branch including a second mating structure configured to mate with the first mating structure to form a substantially fluid tight connection therebetween such that fluid introduced into the third lumen is prevented from exiting the proximal end of the second lumen and is forced distally through the first lumen around the catheter to the distal end of the longitudinal element.

10. The system of claim 9, wherein the shaft includes a hyrdophillic coating.

11. The system of claim 9, further comprising a retention clip including a distal end coupled to the connector distal of the distal end of the second branch, a proximal end of the retention clip being couplable to the hub of the catheter to prevent the hub from being inadvertently separated from the first branch of the connector.

12. The system of claim 11, wherein the retention clip, when coupled to the hub, compresses a distal end of the hub against the proximal end of the first branch of the connector.

13. The system of claim 12, further comprising a compressible member extending around at least a portion of a periphery of the connector between the distal end of the retention clip and the distal end of the second branch.

14. The system of claim 9, wherein the second branch of the connector extends away from the first branch at an angle selected so that a proximal end of the second branch is closer to the proximal end of the first branch than is the distal end of the second branch.

15. The system of claim 9, wherein the longitudinal element of the packaging is flexible.

16. The system of claim 15, wherein the longitudinal element is maintained in a hoop pattern with a length thereof coiled upon itself.

17. The system of claim 9, wherein the hub includes laterally extending wings each with an opposing contour from the other.

18. The system of claim 9, wherein the catheter further comprises a strain relief member coupled to the elongate shaft and the hub.

19. The system of claim 18, wherein the strain relief member comprises a helical coil or spring covered by a tube, wherein the tube is formed over the coil filling the spaces between the individual loops of the helical coil or spring.

20. The packaging of claim 9 wherein the connector is a Y-connector.

21. The packaging of claim 9 wherein the connector is a T-connector.
